(19) United States
(12) Patent Application Publication
(10) Pub. No.: US 2024/0252103 A1
(43) Pub. Date: Aug. 1, 2024

(54) WIRELESS DYSPHAGIA DEVICE

(71) Applicant: Progia Medical Inc., Plymouth, MN (US)

(72) Inventors: Ross Kent Dunbar, Bloomington, MN (US); Gary Lloyd Graham, JR., Plymouth, MN (US); Michael John Brusseau, Center City, MN (US); Luke Wayne Lundquist, Maple Grove, MN (US); James Michael Faucher, St. Michael, MN (US); Kathleen Wendlandt Hill, White Bear Lake, MN (US); Steven Dennis Lind, Rochester, MN (US)

(21) Appl. No.: 18/429,912

(22) Filed: Feb. 1, 2024

Related U.S. Application Data
(60) Provisional application No. 63/442,708, filed on Feb. 1, 2023.

(51) Int. Cl. A61B 5/00; A61B 90/00

(52) U.S. Cl. CPC A61B 5/4542; A61B 5/002; A61B 5/682; A61B 5/6879; A61B 90/06; A61B 2090/064; A61B 2560/0406; A61B 2560/045; A61B 2560/0462; A61B 2562/04; A61B 2562/164

(57) ABSTRACT

A wireless intraoral device for the assessment and treatment of dysphagia includes a molded mouthpiece that is conformable to the patient's mouth and easy for a patient to place and removably secure inside the mouth. The mouthpiece includes a plurality of spaced apart sensors configured to measure tongue pressure. The mouthpiece includes features that improve actuation and performance of the sensors, as well as improve patient comfort and conformity of the mouthpiece inside the mouth. The intraoral device operates wirelessly and all operating components for data measurement and collection can be contained within the intraoral device. The intraoral device can communicate wirelessly with a tablet or other computing device. In an example, the molded mouthpiece can be formed from one or more thermoplastic elastomers, such as silicone.

### CROSS-REFERENCE TO RELATED APPLICATION

This application claims the benefit of U.S. Provisional Application No. 63/442,708, filed on Feb. 1, 2023, which is incorporated by reference herein in its entirety.

### BACKGROUND

Dysphagia is a condition where a person has trouble swallowing food or liquids due to difficulty moving them from the mouth to the stomach. It can result in food or liquids getting stuck in the throat or esophagus and can cause pain or discomfort while swallowing. Dysphagia can be a symptom of underlying medical conditions such as neurological disorders, stroke, or esophageal disorders, and can have serious implications for a person's health if left untreated.

There exist several treatments for dysphagia, the appropriateness of each depending on the underlying cause. Some of these treatments include: dietary modifications (changing the consistency of food or liquids to make swallowing easier), medications (to relax the muscles in the throat or treat underlying conditions that contribute to dysphagia), endoscopic procedures (using a flexible scope to examine the throat and upper esophagus, and to remove any blockages), surgery (in severe cases, surgery may be necessary to correct structural problems in the throat or esophagus), and a variety of therapies that rely on techniques and devices to diagnose and improve muscle control and coordination for swallowing, eating, breathing, and speaking.

Currently, therapeutic and diagnostic tools for the assessment and treatment of dysphagia suffer from certain challenges, both in terms of manufacture and in terms of use. Existing devices are unable to store within the device itself any data generated during use. Moreover, existing devices make use of wired connections, which are cumbersome at best and which can introduce forces that give rise to faulty data. Additionally, existing devices are inflexible, and are incapable of communicating directly with care providers or transferring data to non-dedicated computing devices.

It would be beneficial to develop a dysphagia device that is wireless and easy for a patient to handle and effectively use in the mouth to measure tongue pressure, improve tongue strength, and improve range-of-motion.

### SUMMARY

According to one aspect, an intraoral device configured for placement in the mouth of a subject includes a mouthpiece having a roof side and a tongue side and including a cross-shaped portion. The mouthpiece comprises a first interior part having a cross-shaped component, formed by a first portion and a second portion perpendicular to the first portion, and a plurality of sensors at spaced apart locations on the first and second portions. The mouthpiece further comprises a second interior part having a component that corresponds in size and shape with the first portion of the first interior part. The first and second interior parts are in a stacked configuration inside the mouthpiece such that the first interior part corresponds with the cross-shaped portion of the mouthpiece and is in contact with the tongue side of the mouthpiece, and the second interior part is in contact with the roof side of the mouthpiece, and a second part of the cross-shaped portion of the mouthpiece is more flexible than a first part of the cross-shaped portion of the mouthpiece. The intraoral device further includes a handheld portion connected to the mouthpiece and configured for placing and removably securing the mouthpiece in the mouth of the subject.

According to another aspect, an intraoral device for placement in the mouth of a subject for measuring tongue pressure includes a molded mouthpiece having an anterior end, a posterior end, a first side for placement on a roof of the mouth, a second side opposite the first side, and a molded cross-shaped portion. The molded mouthpiece includes an interior part housed inside the molded mouthpiece, the interior part having a cross-shaped portion and a plurality of sensors on the cross-shaped portion, the plurality of sensors including a back sensor, a front sensor, a right sensor and a left sensor. The intraoral device further includes a plurality of protrusions formed on an exterior of the molded cross-shaped portion of the mouthpiece on the second side of the molded mouthpiece, each protrusion at a location aligned with a corresponding sensor on the cross-shaped portion of the interior part.

According to another aspect, a method of assessing an individual having dysphagia using an intraoral device includes placing the intraoral device in a mouth of the individual. The intraoral device includes a molded mouthpiece having a molded cross-shaped portion formed by a first portion and a second portion perpendicular to the first portion, the molded mouthpiece including an interior component housed inside the molded mouthpiece, the interior component having a cross-shaped portion and a plurality of sensors on the cross-shaped portion, the plurality of sensors including a posterior sensor aligned with the first portion of the molded cross-shaped portion, an anterior sensor aligned with an opposite end of the first portion, a first side sensor aligned with the second part of the molded cross-shaped portion and a second side sensor aligned with an opposite end of the second part. The method further includes adjusting the intraoral device to position the plurality of sensors to contours of a roof of the mouth, the second part of the molded cross-shaped portion being more flexible than the first portion of the molded cross-shaped portion. The method further includes measuring tongue pressure on each of the sensors and collecting data from each of the sensors, and transmitting the data wirelessly to a computing device.

### BRIEF DESCRIPTION OF THE DRAWINGS

In the drawings, which are not necessarily drawn to scale, like numerals may describe similar components in different views. The drawings illustrate generally, by way of example, but not by way of limitation, various embodiments discussed herein.

FIG. 1 is a schematic of an intraoral device for assessing and treating dysphagia.

FIG. 2 is an exploded view of the intraoral device of FIG. 1, rotated approximately 90 degrees counterclockwise.

FIG. 3 is a perspective view of the mouthpiece of the device of FIG. 1.

FIG. 4 is a perspective view of the mouthpiece of FIG. 3, with first and second interior parts shown outside of the mouthpiece.

FIG. 5 is a top view of the first interior part of FIG. 4 including a cross-shaped portion with spaced-apart sensors.

FIG. 6 is a perspective view of a portion of the mouthpiece of FIG. 3.

FIG. 7 is a perspective view of an opposite side of the mouthpiece of FIG. 6 and rotated relative to FIG. 6.

FIG. 8 is a schematic of the intraoral device secured to a mold of upper teeth/jaw.

FIG. 9 is a schematic of the device and mold of FIG. 8 rotated approximately 90 degrees counterclockwise.

FIG. 10 is a schematic of a kit for assessing and treating dysphagia, including the intraoral device of FIG. 1.

### DETAILED DESCRIPTION

The present disclosure is directed to an intraoral device for the assessment and treatment of dysphagia, including improving tongue strength and improving range-of-motion. The intraoral device operates wirelessly and all of the operating components for data measurement and collection can be contained within the intraoral device. The intraoral device can be included in a kit or system that facilitates wireless communication to a tablet or other computing device. The intraoral device includes a mouthpiece that is conformable to the patient's mouth and easy for a patient to place and removably secure inside the mouth. The mouthpiece includes a plurality of spaced apart sensors configured to measure pressure. The mouthpiece includes features that improve actuation and performance of the sensors and overall effectiveness of the device.

The intraoral device can improve range-of-motion in the tongue and during swallowing. Range-of-motion is a critical aspect of treating dysphagia, as the eight muscles that comprise the tongue work in harmony to affect speech, swallowing and breathing. The intraoral device of the present disclosure improves range-of-motion by providing a thin and pliable mouthpiece that can more easily be contoured to the shape of the mouth and is more comfortable for the patient or subject.

The present disclosure includes a method of using the intraoral device to assess and treat an individual having dysphagia. Such method includes placing a molded intraoral device in the mouth of the patient and adjusting the device to position the spaced apart sensors based on the contours of the roof of the mouth. The method includes measuring tongue pressure via the sensors, collecting the measured data from each of the sensors, and transmitting the data wirelessly to a computing device such as a tablet that can be part of a system for use with the device 10.

In some embodiments, the mouthpiece is molded and includes one or more features, such as protrusions or domes on an exterior of the mouthpiece that improve performance of the intraoral device. The molding process for forming the mouthpiece can include the use of one or more elastomers, such as silicone, via a low pressure and low temperature method.

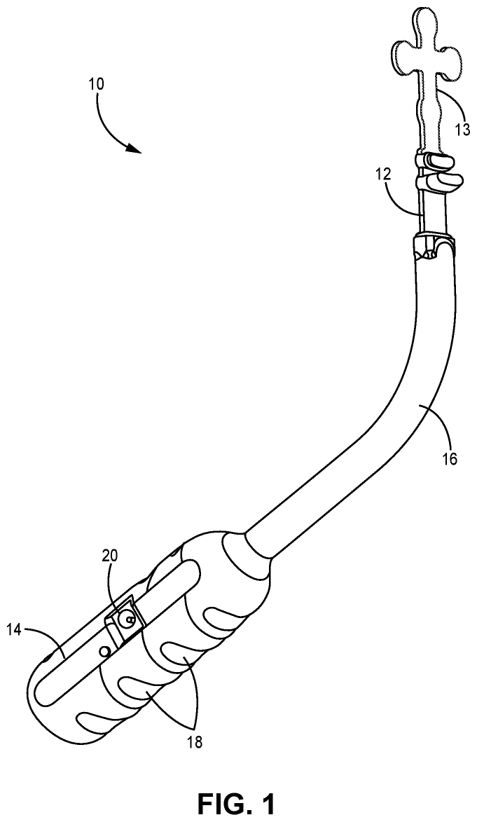

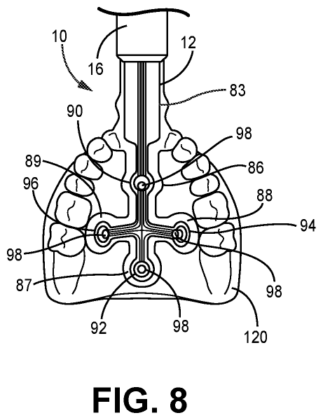

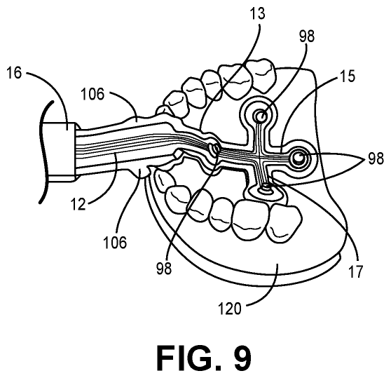

FIG. 1 is a schematic of an intraoral device 10 configured for screening and improving strength generated for swallowing, speech, and breathing. The intraoral device 10 includes a mouthpiece 12, a handle 14, and an extension 16 between the mouthpiece 12 and the handle 14. In some embodiments, the extension 16 can be integrally formed with the handle 14. The mouthpiece 12 is configured for insertion into a mouth of a patient or human subject. The mouthpiece 12 is configured such that a cross-shaped portion 13 of the mouthpiece 12 is placed on the roof or hard palate of the mouth. (See FIGS. 8 and 9 and the corresponding description below.)

The extension 16 can include a curved portion for ease of use. The device 10 is designed such that the handle 14 can comfortably rest on the chest or sternum of the subject when the mouthpiece 12 is inside the mouth of the subject. The handle 14 is designed to accommodate a variety of users and patients, including those with diminished dexterity. The handle 14 can include one or more indentations 18 for ease of use. The device 10 can be wireless and the handle 14 can include a charging port 20. The device 10 is designed such that all of the electrical and electronic functions, as well as the power source, can be incorporated into a single, compact handheld unit that also includes the mouthpiece 12.

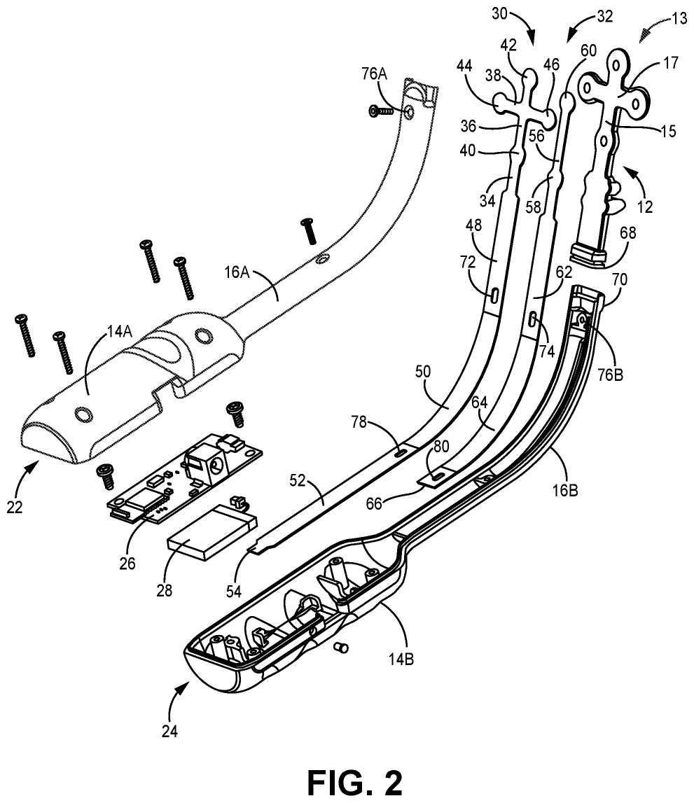

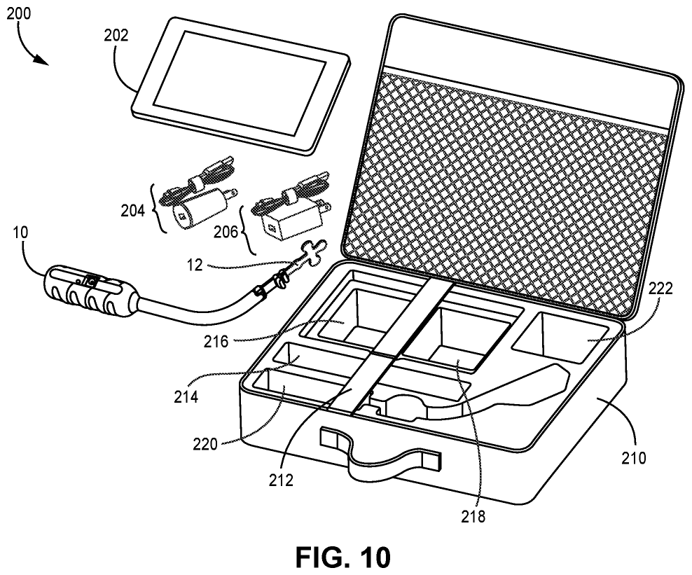

FIG. 2 is an exploded view of the device 10 of FIG. 1. In some embodiments, the handle 14 and extension 16 can be formed of a first half 22 and a second half 24 that fit together. The first half 22 includes a handle portion 14A and an extension portion 16A. The second half 24 includes a handle portion 14B and an extension portion 16B. The handle 14 can house one or more circuit boards 26, 28 for operation of the device 10 and data collection. The device 10 can be configured for wireless operation and wireless communication with one or more computing devices, such as a tablet. This is described further below in reference to FIG. 10. The design of the device 10 and the supporting components shown in FIG. 10 streamlines the interface between patient and clinician by eliminating third-party intervention or management. This eliminates additional costs, lowers barriers for adoption by patients restricted by income or technological knowledge, and adds efficiency to the clinician. The device 10 and supporting components are also designed to keep data out of the cloud, making HIPAA compliance easier.

The mouthpiece 12 includes a first part or first component 30 and a second part or second component 32, both of which are partially housed inside the mouthpiece 12 and partially housed inside the extension 16. (In FIG. 2, the parts 30 and 32 are shown outside the mouthpiece 12 for illustrative purposes only.) The first and second parts 30, 32 are in a stacked configured inside the mouthpiece 12. The first part 30 includes a cross-shaped portion 34 formed by a longitudinal portion 36 (or a first portion) and a latitudinal portion 38 (or a second portion). The first and second portions 36, 38 are generally perpendicular to one another. The longitudinal portion 36 includes two circular regions 40, 42 at opposing ends. The circular region 40 is an anterior region. The circular region 42 is a posterior region. Similarly, the latitudinal portion 38 includes two circular regions 44, 46 at opposing ends. The circular region 44 is a first side region and the circular region 46 is a second side region. Each of the circular regions 40, 42, 44 and 46 is configured to house one or more sensors (described below). For simplicity, the one or more sensors are not shown in FIG. 2 on the part 30.

The first part 30 includes a support portion 48 formed with the cross-shaped portion 34, a first extender portion 50 and a second extender portion 52. The second extender portion 52 is configured to connect to the circuit board 26 via a tail 54.

The second part 32 includes a longitudinal portion 56 that aligns with the longitudinal portion 36 of the first part 30 when the parts 30 and 32 are assembled inside the mouthpiece 12. The second part 32 can be referred to as a spine, a backer or a substrate and is configured to provide structural integrity and strength to the mouthpiece 12. In some embodiments, the second part 32 can include two circular regions 58, 60 that correspond to circular regions 40, 42, respectively, on the longitudinal portion 36 of the first part 30. In some embodiments, the second part 32 is configured to intentionally exclude a corresponding latitudinal portion. In other words, the second part 32 does not include a cross-shaped portion. Because the second part 32 only includes the longitudinal portion 56, the mouthpiece 12 is more malleable or conformable on the sides of the mouthpiece 12. This can make it easier to place the mouthpiece 12 inside the subject's mouth and make it more comfortable for the subject once the mouthpiece 12 is placed on the roof of the mouth.

The second part 32 includes a support portion 62 formed with the longitudinal portion 56, an extender portion 64 and an attachment portion 66.

The cross-shaped portion 13 of the mouthpiece 12 generally matches with a shape of the cross-shaped portion 34 of the first part 30. The cross-shaped portion 13 includes a first portion 15 and a second portion 17 that is perpendicular to the first portion 15. In some embodiments, the mouthpiece 12 is molded and formed of a food grade or medical grade material. In some embodiments, the mouthpiece 12 can be formed of a thermoplastic elastomer (TPE), including a thermoplastic polyutherane (TPU). In some embodiments, the mouthpiece 12 can be formed of silicone. The silicone can make up generally all of the molded mouthpiece or the silicone can be used in combination with one or more other materials.

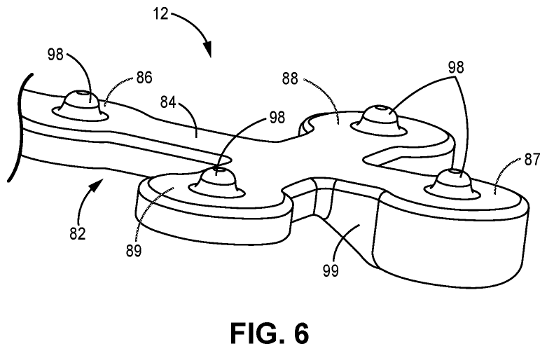

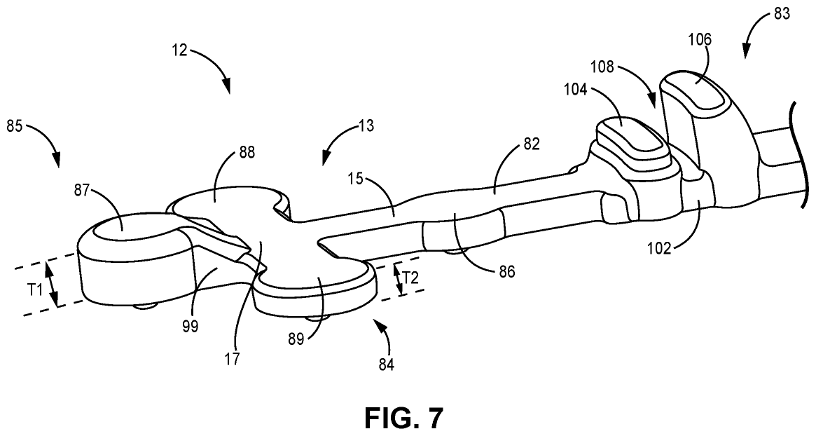

In some embodiments, the mouthpiece 12 is overmolded such that it is formed around the portion of the parts 30 and 32 that are housed inside the mouthpiece 12. One or more features on an exterior of the mouthpiece 12 are designed to improve performance of the sensors inside the mouthpiece 12 (on the first part 30) and improve overall usability and performance of the device 10 for measuring tongue pressure and assessing swallowing functions. See, for example, the description below in reference to FIGS. 6 and 7.

To assemble the device 10, an end feature 68 on the mouthpiece 12 connects to an end 70 of the extension portion 16. An aperture 72 on the support portion 48 and an aperture 74 on the support portion 62 can be aligned with apertures 76A, 76B on the extension portions 16A, 16B, respectively such that a fastener can be used to attach the parts 30, 32 inside the extension portion 16. Similarly, an aperture 78 on the second extender portion 52 and an aperture 80 on the attachment portion 66 can serve as additional attachment points.

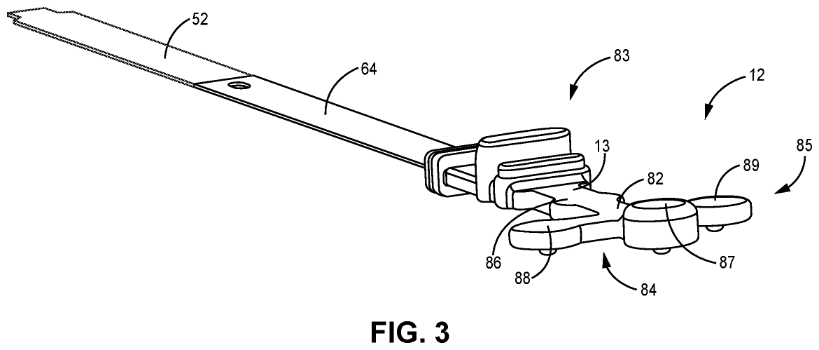

FIG. 3 is a perspective view of the mouthpiece 12, including the first and second parts 30, 32 housed therein. The extender portion 64 of the second part 32 is visible extending from the mouthpiece 12, as well as the second extender portion 52 of the first part 30. The mouthpiece 12 includes a roof side 82 and a tongue side 84 opposite the roof side 82. The mouthpiece 12 includes an anterior end 83 and a posterior end 85. Similar to the first part 30, the cross-shaped portion 13 of the mouthpiece 12 includes circular regions 86, 87, 88 and 89 that align with or correspond to circular regions 40, 42, 44 and 46 on the first part 30 for housing the sensors.

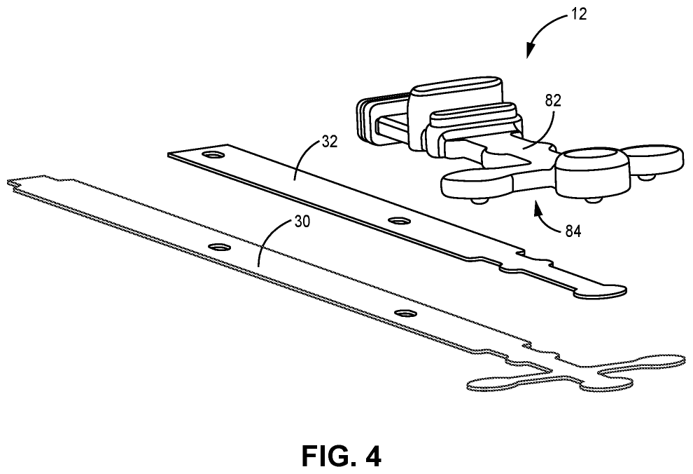

FIG. 4 shows the mouthpiece 12 of FIG. 3 with the parts 30 and 32 outside of the mouthpiece 12 for illustrative purposes only. FIG. 4 shows how the parts 30 and 32 are configured to be stacked inside the mouthpiece 12. The part 30 is in proximity to the tongue side 84 of the mouthpiece 12 while the part 32 is in proximity to the roof side 82. The sensors on the part 30 are not visible in FIG. 4 because they are on the underside of the part 30 as it is oriented in FIG. 4.

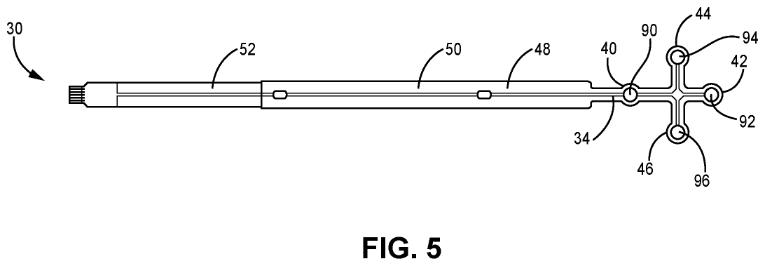

FIG. 5 is a top view of the part 30. The part 30 is flipped over, relative to how the part 30 is shown in FIG. 4. FIG. 5 shows the side of the part 30 in proximity to the tongue side 84 of the mouthpiece 12. As previously described in reference to FIG. 2, the part 30 includes the cross-shaped portion 34, the support portion 48, the first extender portion 50 and the second extender portion 52. The part 30 includes four sensors at spaced apart locations on the cross-shaped portion 34. More specifically, a sensor is located on each of the circular regions 40, 42, 44 and 46 on the cross-shaped portion 34. An anterior sensor 90 is on the anterior circular region 40. A posterior sensor 92 is on the posterior circular region 42. A first side sensor 94 is on the first side circular region 44 and a second side sensor 96 is on the second side circular region 46. Side sensors 94 and 96 can also be referred to as being on the left or right side.

In some embodiments, the sensors 90, 92, 94 and 96 are force sensing resistors (FSR) that exhibit a decrease in resistance when increased force is applied to the sensor. As such, these pressure sensors can measure lingual pressure applied to the sensor by the user's tongue. The sensors 90, 92, 94 and 96 are low cost, ultra-thin, robust and simple to use in the intraoral device 10. In some embodiments, the sensors 90, 92, 94 and 96 on the part 30 can be part of an FSR assembly and the part 30 can include multiple layers (including circuitry) for operation of the sensors 90, 92, 94 and 96.

FIG. 6 shows a portion of the mouthpiece 12 with the tongue side 84 visible. The tongue side 84 can include a plurality of domes or protrusions 98 that correspond in location with the sensors 90, 92, 94 and 96 on the first part 30. The mouthpiece 12 can include a riser 99 on the roof side 82. The riser 99 is described further below in reference to FIG. 7.

As shown in FIG. 6, the protrusions 98 extend from each of the circular regions 86, 87, 88 and 89 on the cross-shaped portion 13 of the mouthpiece 12. In some embodiments, the protrusions 98 are a dome shape. The protrusions/domes 98 can improve sensor activation precision, as well as the ability for the user or patient to locate the sensor and its position in the mouth. Given that the mouthpiece 12 can be molded, the domes 98 can easily be formed as an integral part of the mouthpiece 12. Having the domes 98 integral to the cross-shaped portion 13 of the mouthpiece 12 prevents or minimizes a possibility of the domes 98 becoming detached from the cross-shaped portion 13. It would be undesirable for the domes 98 to detach or separate from the mouthpiece 12, particularly when the mouthpiece 12 is inside the mouth of the subject. In other embodiments, the domes 98 can be a separate component that can be attached to the tongue side 84 of the mouthpiece 12.

In some embodiments, the domes 98 have a height ranging between about 0.05 inches and about 0.08 inches. In some embodiments, the domes 98 have a height of about 0.06 inches. In some embodiments, the domes have a diameter of about 0.1 inches.

It is recognized that the protrusions 98 do not have to be dome shaped. In other embodiments, the protrusions 98 can be formed from a different shaped feature on the tongue side 84 of the mouthpiece 12 that aligns with the location of the sensors 90, 92, 94 and 96. Because the mouthpiece 12 can be formed from a mold, there is flexibility and diversity in the shape and size of the feature that forms the protrusions 98.

FIG. 7 shows a larger portion of the mouthpiece 12 (relative to FIG. 6) with the roof side 82 visible. The riser 99 is shown near the posterior end 85 of the cross-shaped component 13 of the mouthpiece 12. The posterior end 85 of the mouthpiece 12 is configured for placement towards the back of the mouth of the subject. The riser 99 is designed such that the tongue side 84 of the mouthpiece 12 at the posterior end 85 is dropped down (closer to the tongue) when the mouthpiece 12 is in the mouth of the subject or patient. It may be difficult in some cases for the patient to reach the posterior sensor 92 (corresponding to the circular region 87) if the posterior sensor 92 is too close to the roof of the mouth and thus too far from the tongue. The riser 99 makes the posterior circular region 87 more accessible to the tongue.

In some embodiments, the riser 99 is formed by incrementally increasing a thickness of the mouthpiece 12, specifically at or around the circular region 87 at the posterior end 85 of the mouthpiece 12. As shown in FIG. 7, the thickness of the mouthpiece 13 on the first portion 15 increases from the second part 17 to the circular region 87, thus creating a ramp (i.e. riser 99) to the circular region 87. This results in the circular region 87 having a thickness T1 that is greater than a thickness T2 of the circular regions 86, 88 and 89.

In some embodiments, the riser 99 is an integral part of the molded mouthpiece 12 and it is not separate from the rest of the cross-shaped portion 13. Similarly, in some embodiments, the circular region 87 is integral and formed to be of greater thickness when the mouthpiece 12 is made. In other embodiments, the riser 99 could be added onto the first portion 15 between the second part 17 and the circular region 87, along with an extension piece added onto the circular region 87 to increase the thickness of the circular region 87.

As described above, the thickness T1 is greater than the thickness T2. In some embodiments, the thickness T1 is about two times greater than the thickness T2. In other embodiments, the thickness T1 is more than two times greater than the thickness T2. In some embodiments, the thickness T2 is between about 0.09 and about 0.12 inches. In some embodiments, the thickness T2 is about 0.11 inches. In some embodiments, the thickness T1 is between about 0.2 and about 0.25 inches. In some embodiments, the thickness T1 is about 0.23 inches. The other areas of the cross-shaped portion 13 of the mouthpiece 12 can generally have a thickness similar to the thickness T2. The mouthpiece 12 is designed to include the structural features described herein that facilitate or improve performance of the mouthpiece 12 for measuring tongue pressure, while minimizing an overall shape and thickness of the mouthpiece 12 for patient comfort and usability.

In some embodiments, the posterior circular region 87 can have a slight dome shape on the roof side 82. In other embodiments, the posterior circular region 87 can have a generally flat surface on both the roof side 82 and the tongue side 84. Other structural features, in addition to or as an alternative to the riser 99 and the thicker posterior circular region 87, can be used so that the posterior end 85 of the mouthpiece 12 extends further down from the roof of the mouth. The other portions of the mouthpiece 12 are configured to be thinner to prevent the mouthpiece 12 from being bulky and to maintain pliability and conformability of the mouthpiece 12 inside the mouth.

In an example, a size of the mouthpiece 12 can be defined as the widest distance (end to end) between the circular side portions 88 and 89. In some embodiments, such distance can range between about 1.25 inches and about 1.75 inches. In some embodiments, such distance can be about 1.5 inches.

The mouthpiece 12 includes a bite locator 102 formed from a first bump or ridge 104 and a second bump or ridge 106, and the two ridges 104, 106 are separated by a cavity 108. The second ridge 106 is located anterior to the first ridge 104. In some embodiments, the second ridge 106 has a greater height or thickness relative to the first ridge 104. The bite locator 102 can be used to aid in placement and/or securement of the mouthpiece 12 in the mouth.

The bite locator 102 is designed to accommodate different mouth sizes and patient scenarios. In one scenario, the subject can put their front teeth in the cavity 108 of the bite locator 102. For an average size mouth, this results in proper anterior/posterior placement of the mouthpiece 12 inside the mouth. In a second scenario, if the subject puts their front teeth in the cavity 108 and the mouthpiece 12 is not far enough back in the mouth, the subject can alternatively place their front teeth posterior to the first ridge 104. In a third scenario, if the subject does not have teeth or dentures, the subject can rest their gums on the first ridge 104. Even thought the bite locator 102 is fixed in place on the mouthpiece 12, the design of the bite locator 102 provides flexibility to the subject, depending on their individual needs or anatomy.

The bite locator 102 allows for a stable, secure positioning of the mouthpiece 12 within the mouth of the subject. It also facilitates repeatable placement and measurement. Once the subject knows where to place their teeth or gums on the bite locator 102, the subject can repeat that placement during subsequent uses of the intraoral device 10. Consistent placement of the device 10 in the mouth helps achieve consistent measurements over time.

FIG. 8 is a schematic of the device 10 secured on a mold 120 of an upper jaw/teeth to illustrate how the device 10 is placed in the mouth of a subject. FIG. 9 is a schematic of the device 10 and the mold 120 of FIG. 8 rotated approximately 90 degrees counterclockwise. Specifically, FIGS. 8 and 9 show the mouthpiece 12 secured to the mold 120 and a portion of the extension 16 of the device 10 extending from the anterior end 83 of the mouthpiece 12.

FIGS. 8 and 9 show the circular regions 86, 87, 88 and 89 on the mouthpiece 12, including the domes or protrusions 98 in a center of the circular regions 86, 87, 88 and 89. The sensors 90, 92, 94 and 96 can also be seen under the protrusions 98. The protrusions 98 serve as a guide for the user's tongue to locate the sensors 90, 92, 94 and 96. The position of the protrusions 98 in the center of the circular regions 86, 87, 88 and 89 improves the functionality of the sensors 90, 92, 94 and 96 by displacing the load across the full diameter of the underlying sensor.

The mouthpiece 12 is configured for the posterior end 85 to be at the back of the mouth and the anterior end 83 to be in proximity to the front teeth or gumline of the mouth. The first portion 15 of the cross-shaped portion 13 of the mouthpiece 12 is positioned between the back of the mouth and the gumline and the second part 17 of the cross-shaped portion is positioned between the sides of the gumline. The roof side 82 of the mouthpiece 12 can be removably secured to the hard palate or roof of the mouth. FIGS. 8 and 9 illustrate how the mouthpiece 12 is malleable and bendable and conforms to curvature in the mouth and the shape of the roof of the mouth. Because the second part 32 only extends in an anterior-posterior direction, corresponding with the first portion 15 of the molded mouthpiece, the second part 17 of the molded mouthpiece is more flexible than the first portion 15 and helps with placement of the mouthpiece 12 in the mouth and with user comfort. The front teeth on the mold 120 are insertable between the first ridge 104 and the second ridge 106.

As provided above, the mouthpiece 12 can be a molded mouthpiece. In some embodiments, the mouthpiece 12 can be overmolded. Challenges can arise in the molding process given the inclusion of the part 30 (which includes the sensors 90, 92, 94 and 96) and the part 32 inside the mold. The mouthpiece 12 can be formed using overmolding at low temperatures and pressures to maintain the integrity and functionality of the sensors 90, 92, 94, and 96. In some embodiments, the mouthpiece 12 is formed of silicone.

The intraoral device 10 can improve range-of-motion in the tongue and during swallowing via the design of the mouthpiece 12, which can easily be inserted into the mouth and conform to the shape of the mouth. The second part 32 of the mouthpiece 12 can be thinner. Since the second part 32 does not include a latitudinal portion, the cross-shaped portion 13 of the mouthpiece 12 can be more pliable or flexible on the sides of the mouthpiece 13. This helps with conforming the mouthpiece 13 to match the contours of the subject's mouth. Moreover, the molded mouthpiece 12 can be formed of a material, such as silicone, that is sufficiently pliable. An overall thickness of the molded mouthpiece 12 can be reduced or minimized to also aid in the pliability of the mouthpiece 12.

FIG. 10 is a schematic of a kit or system 200 for assessing and treating dysphagia. The kit or system 200 includes the device 10, a tablet 202, a first charger 204 for the device 10 and a second charger 206 for the tablet 202. The kit or system 200 also includes a case 210 to store and carry the components of the kit 200. As shown in FIG. 10, the case 210 includes various compartments to hold or house the components and a fastener 212, such as hook and loop, to secure the components inside the compartments and inside the case 210.

The tablet 202 can receive the data measured and collected by the intraoral device 10. The tablet 202 can display the data such that it can be used by the patient or doctor for assessment and potential treatment. Other computing devices can be used as an alternative to or in addition to the tablet 202. Such devices can include, but are not limited to, a computer or a phone.

The case 210 can include a slot or elongated compartment 214 configured to serve as an easel such that the tablet 202 can be held upright inside the case 210 during use of the kit 200. In an example, the tablet 202 can be used in real time to capture data measured and collected by the intraoral device 10. The tablet 202 can be used by the patient and/or a clinician, or in some cases by a caregiver. Because the tablet 202 can be used in real time, the patient and clinician can receive immediate biofeedback. Goals can be set to increase tongue strength. The data and goals can be stored in the tablet 202. The kit 200 is portable such that the kit 200 can be used at home, at a medical clinic or another location. The clinician can review the data on the tablet 202 and observe trends and modify treatment accordingly.

When the kit 200 is not in use, the chargers 204, 206 can be stored in compartments 216 and 218 and the tablet 202 can sit atop the compartments 216 and 218 and be secured in place via the fastener 212.

A compartment 220 is configured to receive the intraoral device. Although not shown in FIG. 10, the kit 200 can include a clamshell or similar type of small container that can house the mouthpiece 12 when the device 10 is not in use. An extra compartment 222 can be used to house additional components of the kit 200, such as, for example, a small towel or towelette.

The system 200 uses wireless technology, such as Bluetooth. Previous designs maintained a separate, wired, digitizer device that the intraoral device had to be plugged into to interface with a tablet or a computer. With the design of the intraoral device 10 disclosed herein, all functions, electronic and physical, are in a single device that communicates with the tablet, computer or phone through Bluetooth Low Energy (BLE) or other wireless technology.

Operation of the system 200 is streamlined, including the entire communication architecture, eliminating the administrator and all “cloud” or third-party storage service. By streamlining the interaction between the patient and clinician and eliminating a third-party source, the structure required to assure HIPAA compliance is not needed. This also eliminates the practice itself of using the third party source, including a subscription to support the third party. This can make it easier to work with medical providers, such as, for example, the VA (Veteran Affairs).

In some embodiments, operation of the system 200 can include communications between the clinician and patient through automated emails. For example, emails can be automated to be sent nightly at midnight, thus removing any need for the patient to remember or for the clinician to pursue the date. In dysphagia therapy and force measurement, time from data measurement to clinician review is less urgent than in other medical recordings.

The system 200 can be designed to operate as a stand alone system. The system can also integrate with video conferencing, such as, for example, TeleHealth, such that the clinician can train, measure, guide and counsel the patient virtually or remotely. The system 200 can also be used with applications specifically developed for the system 200 or with other smart phone-based apps or other similar devices (iPads, etc.).

While the invention has been described with reference to an exemplary embodiment(s), it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment(s) disclosed, but that the invention will include all embodiments falling within the scope of the appended claims.

### Discussion of Possible Embodiments

The following are non-exclusive descriptions of possible embodiments of the present invention.

According to one aspect, an intraoral device configured for placement in the mouth of a subject includes a mouthpiece having a roof side and a tongue side and including a cross-shaped portion. The mouthpiece further includes a first interior part having a cross-shaped component, formed by a first portion and a second portion perpendicular to the first portion, and a plurality of sensors at spaced apart locations on the first and second portions. The mouthpiece further includes a second interior part having a component that corresponds in size and shape with the first portion of the first interior part. The first and second interior parts are in a stacked configuration inside the mouthpiece such that the first interior part corresponds with the cross-shaped portion of the mouthpiece and is in contact with the tongue side of the mouthpiece, and the second interior part is in contact with the roof side of the mouthpiece, and a second part of the cross-shaped portion of the mouthpiece is more flexible than a first part of the cross-shaped portion of the mouthpiece. The intraoral device further includes a handheld portion connected to the mouthpiece and configured for placing and removably securing the mouthpiece in the mouth of the subject.

The device of the preceding paragraph can optionally include, additionally and/or alternatively any, one or more of the following features, steps, configurations and/or additional components.

In some embodiments, an extension piece connects the handheld portion to the mouthpiece and the extension piece includes a curved portion.

In some embodiments, the plurality of sensors comprises an anterior sensor on an anterior end of the first portion of the cross-shaped component of the first interior part and a posterior sensor on a posterior end of the first portion of the cross-shaped component of the first interior part.

In some embodiments, the plurality of sensors comprises a left sensor on the second portion of the cross-shaped component of the first interior part and a right sensor opposite of the left sensor on the second portion of the cross-shaped component of the first interior part.

In some embodiments, the plurality of sensors include force sensing resistors.

In some embodiments, the mouthpiece is formed of a thermoplastic elastomer.

In some embodiments, the mouthpiece is formed of silicone.

In some embodiments, the mouthpiece is formed by a molding process.

In some embodiments, the molded mouthpiece includes a plurality of protrusions formed on an exterior of the molded mouthpiece on the tongue side, and a location of each protrusion on the molded mouthpiece corresponds with a location of a sensor on the cross-shaped component of the first interior part.

In some embodiments, the protrusions are dome shaped.

In some embodiments, a thickness of the mouthpiece at a posterior end of the mouthpiece is greater than a thickness of the mouthpiece at an anterior end of the mouthpiece and the mouthpiece includes a ramp on the first part of the cross-shaped portion of the mouthpiece near the posterior end.

In some embodiments, the mouthpiece includes a bite locator at an anterior end of the mouthpiece, the bite locator comprises a first ridge and a second ridge separated by a cavity.

According to another aspect, an intraoral device for placement in the mouth of a subject for measuring tongue pressure includes a molded mouthpiece having an anterior end, a posterior end, a first side for placement on a roof of the mouth, a second side opposite the first side, and a molded cross-shaped portion, the molded mouthpiece including an interior part housed inside the molded mouthpiece, the interior part having a cross-shaped portion and a plurality of sensors on the cross-shaped portion, the plurality of sensors including a back sensor, a front sensor, a right sensor and a left sensor. The intraoral device further includes a plurality of protrusions formed on an exterior of the molded cross-shaped portion of the mouthpiece on the second side of the molded mouthpiece, each protrusion at a location aligned with a corresponding sensor on the cross-shaped portion of the interior part.

The device of the preceding paragraph can optionally include, additionally and/or alternatively any, one or more of the following features, steps, configurations and/or additional components.

In some embodiments, the protrusions are dome shaped.

In some embodiments, the molded mouthpiece further comprises a metal support part housed inside the molded mouthpiece between the interior part and the first side of the molded mouthpiece and in a stacked configuration with the interior part.

In some embodiments, the cross-shaped portion of the interior part comprises a longitudinal portion, extending from the anterior end of the mouthpiece towards the posterior end of the mouthpiece, and a latitudinal portion generally perpendicular to the longitudinal portion.

In some embodiments, a size and shape of the metal support part is similar to the longitudinal portion of the interior part such that a second part of the molded cross-shaped portion of the molded mouthpiece is more flexible relative to a first part of the molded cross-shaped portion.

In some embodiments, the molded cross-shaped portion of the molded mouthpiece includes a posterior circular region and an anterior circular region, and a thickness of the posterior circular region is greater than a thickness of the anterior circular region.

In some embodiments, a posterior protrusion in the plurality of protrusions is positioned in a center of the posterior circular region and an anterior protrusion in the plurality of protrusions is positioned in a center of the anterior circular region.

In some embodiments, the molded mouthpiece is formed of one or more thermoplastic elastomers.

In some embodiments, the molded mouthpiece is formed of silicone.

In some embodiments, the device further comprises a handle to aid in placing and removably securing the mouthpiece inside the mouth.

In some embodiments, the device further comprises an extender connecting the handle to the mouthpiece, wherein the extender is curved.

In some embodiments, the device operates wirelessly and is configured to communicate data to a tablet or computing device.

According to another aspect, a method of assessing an individual having dysphagia using an intraoral device includes placing the intraoral device in a mouth of the individual. The intraoral device includes a molded mouthpiece having a molded cross-shaped portion formed by a first portion and a second portion perpendicular to the first portion, the molded mouthpiece including an interior component housed inside the molded mouthpiece, the interior component having a cross-shaped portion and a plurality of sensors on the cross-shaped portion, the plurality of sensors including a posterior sensor aligned with the first portion of the molded cross-shaped portion, an anterior sensor aligned with an opposite end of the first portion, a first side sensor aligned with the second part of the molded cross-shaped portion and a second side sensor aligned with an opposite end of the second part. The method further includes adjusting the intraoral device to position the plurality of sensors to contours of a roof of the mouth, the second part of the molded cross-shaped portion being more flexible than the first portion of the molded cross-shaped portion. The method further includes measuring tongue pressure on each of the sensors and collecting data from each of the sensors, and transmitting the data wirelessly to a computing device.

The method of the preceding paragraph can optionally include, additionally and/or alternatively any, one or more of the following features, steps, configurations and/or additional components.

In some embodiments, the intraoral device comprises a bite locator configured to receive the individual's teeth or gums during placement of the intraoral device in the mouth.

In some embodiments, the bite locator includes a first ridge and a second ridge at an anterior end of the molded mouthpiece, and a cavity separates the first ridge from the second ridge.

In some embodiments, the method further comprises placing the teeth in the cavity and biting down on the mouthpiece.

In some embodiments, the method further comprises, if placement of the teeth in the cavity results in the device not being far enough back in the mouth, removing the teeth from the cavity and placing the teeth posterior to the first ridge and biting down on the mouthpiece.

In some embodiments, the intraoral device further comprises a plurality of protrusions formed on an exterior of the molded cross-shaped portion of the mouthpiece, each protrusion at a location aligned with a corresponding sensor on the cross-shaped portion of the interior part.

In some embodiments, the intraoral device further comprises a handheld portion for the individual to grip while using the device, and the method further comprising resting the handle on a sternum of the individual.

In some embodiments, the intraoral device and the computing device are part of a kit that is contained within a portable case.

1. An intraoral device configured for placement in the mouth of a subject, the device comprising:
a mouthpiece having a roof side and a tongue side and including a cross-shaped portion, the mouthpiece comprising:
a first interior part having a cross-shaped component, formed by a first portion and a second portion perpendicular to the first portion, and a plurality of sensors at spaced apart locations on the first and second portions; and
a second interior part having a component that corresponds in size and shape with the first portion of the first interior part,
wherein the first and second interior parts are in a stacked configuration inside the mouthpiece such that the first interior part corresponds with the cross-shaped portion of the mouthpiece and is in contact with the tongue side of the mouthpiece, and the second interior part is in contact with the roof side of the mouthpiece, and a second part of the cross-shaped portion of the mouthpiece is more flexible than a first part of the cross-shaped portion of the mouthpiece; and
a handheld portion connected to the mouthpiece and configured for placing and removably securing the mouthpiece in the mouth of the subject.

2. The intraoral device of claim 1, wherein an extension piece connects the handheld portion to the mouthpiece and the extension piece includes a curved portion.

3. The intraoral device of claim 1, wherein the plurality of sensors comprises an anterior sensor on an anterior end of the first portion of the cross-shaped component of the first interior part and a posterior sensor on a posterior end of the first portion of the cross-shaped component of the first interior part.

4. The intraoral device of claim 3, wherein the plurality of sensors comprises a left sensor on the second portion of the cross-shaped component of the first interior part and a right sensor opposite of the left sensor on the second portion of the cross-shaped component of the first interior part.

5. The intraoral device of claim 1, wherein the plurality of sensors include force sensing resistors.

6. The intraoral device of claim 1, wherein the mouthpiece is formed of a thermoplastic elastomer.

7. The intraoral device of claim 1, wherein the mouthpiece is formed by a molding process.

8. The intraoral device of claim 7, wherein the molded mouthpiece includes a plurality of protrusions formed on an exterior of the molded mouthpiece on the tongue side, and a location of each protrusion on the molded mouthpiece corresponds with a location of a sensor on the cross-shaped component of the first interior part.

9. The intraoral device of claim 8, wherein the protrusions are dome shaped.

10. The intraoral device of claim 1, wherein a thickness of the mouthpiece at a posterior end of the mouthpiece is greater than a thickness of the mouthpiece at an anterior end of the mouthpiece and the mouthpiece includes a ramp on the first part of the cross-shaped portion of the mouthpiece near the posterior end.

11. The intraoral device of claim 1, wherein the mouthpiece includes a bite locator at an anterior end of the mouthpiece, the bite locator comprises a first ridge and a second ridge separated by a cavity.

12. An intraoral device for placement in the mouth of a subject for measuring tongue pressure, the device comprising:
a molded mouthpiece having an anterior end, a posterior end, a first side for placement on a roof of the mouth, a second side opposite the first side, and a molded cross-shaped portion, the molded mouthpiece including an interior part housed inside the molded mouthpiece, the interior part having a cross-shaped portion and a plurality of sensors on the cross-shaped portion, the plurality of sensors including a back sensor, a front sensor, a right sensor and a left sensor; and
a plurality of protrusions formed on an exterior of the molded cross-shaped portion of the mouthpiece on the second side of the molded mouthpiece, each protrusion at a location aligned with a corresponding sensor on the cross-shaped portion of the interior part.

13. The intraoral device of claim 12, the molded mouthpiece further comprising a metal support part housed inside the molded mouthpiece between the interior part and the first side of the molded mouthpiece and in a stacked configuration with the interior part.

14. The intraoral device of claim 13, wherein the cross-shaped portion of the interior part comprises a longitudinal portion, extending from the anterior end of the mouthpiece towards the posterior end of the mouthpiece, and a latitudinal portion generally perpendicular to the longitudinal portion.

15. The intraoral device of claim 14, wherein a size and shape of the metal support part is similar to the longitudinal portion of the interior part such that a second part of the molded cross-shaped portion of the molded mouthpiece is more flexible relative to a first part of the molded cross-shaped portion.

16. The intraoral device of claim 12, wherein the device operates wirelessly and is configured to communicate data to a tablet or computing device.

17. A method of assessing an individual having dysphagia using an intraoral device, the method comprising:
placing the intraoral device in a mouth of the individual, the intraoral device comprising:
a molded mouthpiece having a molded cross-shaped portion formed by a first portion and a second portion perpendicular to the first portion, the molded mouthpiece including an interior component housed inside the molded mouthpiece, the interior component having a cross-shaped portion and a plurality of sensors on the cross-shaped portion, the plurality of sensors including a posterior sensor aligned with the first portion of the molded cross-shaped portion, an anterior sensor aligned with an opposite end of the first portion, a first side sensor aligned with the second part of the molded cross-shaped portion and a second side sensor aligned with an opposite end of the second part;
adjusting the intraoral device to position the plurality of sensors to contours of a roof of the mouth, the second part of the molded cross-shaped portion being more flexible than the first portion of the molded cross-shaped portion;
measuring tongue pressure on each of the sensors and collecting data from each of the sensors; and
transmitting the data wirelessly to a computing device.

18. The method of claim 17, wherein the intraoral device comprises a bite locator configured to receive the individual's teeth or gums during placement of the intraoral device in the mouth.

19. The method of claim 17, wherein the bite locator includes a first ridge and a second ridge at an anterior end of the molded mouthpiece, and a cavity separates the first ridge from the second ridge.

20. The method of claim 17, the intraoral device further comprising: a plurality of protrusions formed on an exterior of the molded cross-shaped portion of the mouthpiece, each protrusion at a location aligned with a corresponding sensor on the cross-shaped portion of the interior part.
